(19) United States
(12) Patent Application Publication
(10) Pub. No.: US 2012/0083037 A1
(43) Pub. Date: Apr. 5, 2012

(54) Non-Viral Transfection Agent

(75) Inventors: Joachim H. Wendorff, Nauheim (DE); Achim Aigner, Marburg (DE); Ronald Dersch, Marburg (DE); Sabrina Höbel, Marburg (DE); Markus Rudisile, Heidelberg (DE)

(73) Assignee: Philipps Universitat Marburg, Marburg (DE)

(21) Appl. No.: 13/145,756

(22) PCT Filed: Jan. 29, 2010
(86) PCT No.: PCT/EP2010/051081

§ 371 Date: Dec. 15, 2011

(30) Foreign Application Priority Data
Jan. 29, 2009 (DE) 102009006606.3

(51) Int. Cl. C12N 15/85; B29C 47/00

(52) U.S. Cl. 435/455; 435/320.1; 264/465

(57) ABSTRACT

The invention relates to a non-viral transfection agent comprising polymer/nucleic acid complexes and nanofibers, wherein the polymer/nucleic acid complexes are composed of at least one nucleic acid and at least one cationic polymer. The nanofibers carry the polymer/nucleic acid complexes, wherein the non-viral transfection agent is advantageously produced by means of electrospinning. The cationic polymer is favorably a polyimine or polyethyleneimine and can be modified with one or more hydrophilic polymers coupled thereto. It can also be advantageous to couple the cationic polymer with one or more carbohydrates and/or with a receptor-specific ligand. The nucleic acid is a DNA or an RNA, or a DNA or RNA derivative, advantageously a therapeutically active nucleic acid. The nanofibers are composed of biodegradable, biocompatible polymers. The nanofibers or the entire transfection agent can be provided with a polymer coating. A method for producing a non-viral transfection agent comprises the following steps: providing the polymer/nucleic acid complex, producing a spinning solution containing the polymer/nucleic acid complexes, and electrospinning.

The invention concerns a non-viral transfection agent according to claim 1, a method for its production according to claim 17, the use according to claims 20 and 21, as well as a pharmaceutical comprising a non-viral transfection agent and its use according to claims 22 and 23.

Transfection agents are generally known and are becoming increasingly important in the area of gene therapies, especially with regard to the use of nucleic acid molecules. Their aim is to transport nucleic acid molecules based on DNA (e.g. expression plasmids)—though this increasingly involves the transportation of short-chain nucleic acid molecules based on RNA (e.g. siRNAs) as well—into the cells of a culture or target tissue to be treated.

Physical or chemical methods or virus-based systems such as modified adeno- or retroviruses are usually used to transfect eukaryotic cells with DNA plasmids in a stable and transient manner. Whereas most of the physical or chemical methods—particularly in the case of mammalian cells—are often not very effective and rather difficult to be applied to the entire organism, the virus-based systems normally comprise a relatively high efficiency. However, they also have crucial disadvantages. On the one hand, they are very time-consuming during the preparation and highly sensitive in their handling. On the other hand, they involve substantial risks, in particular with regard to the risk of immune reactions within the immune system or oncogenetic diseases.

Thus, attempts have been made to develop non-viral transfection agents. As such, WO 96/02655 A1 describes, by way of example, polycation/DNA complexes for the in-vivo transfer of DNA. However, these complexes show a drastically reduced transfection efficiency in comparison to viral transfection agents. This often correlates with non-specific interactions of the polycation/DNA complexes—for instance with blood components—and aggregate formations.

In order to prevent the latter from occurring in particular, the polycation/DNA complexes were further developed in WO 98/59064 A1 by modifying the cationic polymers—especially polyethyleneimine (PEI). The aim of this development was to successfully shield the cationic polymer surface and therefore impede the aggregate formation.

However, it was found that relatively high molecular weights of the modified PEI are required for the production of these complexes. This subsequently leads to sterically small polymer/nucleic acid complexes. Unfortunately, however, the smaller the complexes are, the more reduced the transfection efficiency seems to be. It is therefore necessary to use a higher concentration of the complexes; this, in turn, favors the aggregate formation.

Furthermore, the storage of the complexes is problematic. Although the complexes are suitable for storage over a limited time period in the solution, this again involves the risk of aggregation and subsequently an enormous reduction in the transfection efficiency. In addition, storage is only possible in lyophilized or deep-frozen form in a few cases. However, this is connected with further substantial preparation effort. Furthermore, this does not necessarily ensure that the preparation comprises a similar bio-activity as the freshly produced preparation after re-dissolution or thawing.

Within the context of the upcoming and promising possibilities in the area of gene therapies, it has also been of major interest for some time to introduce not only DNA, but also therapeutically effective RNA into mammalian cells. Therapeutically effective RNA are significantly more critical than DNA in handling; viral systems are only suitable for use in a limited manner. As such, it is not possible, for example, to use them for the transport of siRNAs. WO 01/43777 A1 therefore provides the complexation of RNA, in particular of ribozymes, with PEI to polycation/RNA complexes. With these complexes, the aforementioned problem of aggregate formation is, inter alia, present here too. Another significant problem is that it is either very difficult or even impossible to store these complexes.

That a loco-regional and above all temporally controlled release of the nucleic acids is very difficult is inherent to these solutions. Thus, the loco-regional and above all temporal control of the cellular uptake of polymer/nucleic acid complexes is very difficult.

Furthermore, all these systems are only usable in the form of aqueous or organic solutions. This, in turn, causes the dosage of the nucleic acids to be difficult and imprecise to measure. This also applies with regard to the risk of aggregation. In particular, these systems do not allow for a combination of sufficiently efficient uptake into the target cells of the target tissue and simultaneously sufficiently loco-regionally and temporally controllable release of the nucleic acids into the target tissue.

The invention therefore aims to provide a non-viral transfection agent which overcomes the aforementioned disadvantages. In particular, it has to facilitate the protection of the nucleic acids against degradation and their efficient introduction into tissue and cells, as well as an improved loco-regionally controlled and above all temporally controlled release of the nucleic acids. Furthermore, the transfection agent has to be able to be stored simply and suitable to be produced with the simplest possible means and at a reasonable price.

The main characteristics of the invention are indicated in claims 1, 12 and 13 to 16. Arrangements are the subject of claims 2 to 11.

The invention provides a non-viral transfection agent, comprising polymer/nucleic acid complexes and nanofibers, wherein the polymer/nucleic acid complexes comprise at least one nucleic acid and at least one cationic polymer.

Furthermore, the invention provides a method for the production of a non-viral transfection agent, comprising the steps
provision of the polymer/nucleic acid complex
production of a spinning solution, comprising the polymer/nucleic acid complexes and at least one carrier polymer
electrospinning.

A major advantage of the non-viral transfection agent according to the present invention is that it comprises nanofibers which carry the polymer/nucleic acid complexes. As a result of this, the aggregation of the complexes in particular is prevented, and the transfection agent is suitable for storage without problems. By way of example, the dry nanofibers with the polymer/nucleic acid complexes are suitable for storage at 4° C. for several weeks without problems and without the loss of bio-activity.

A further advantage of the invention is that the complexes are suitable to be released from the nanofibers in a protracted manner. As a result of this, the loco-regional and temporal control of the uptake of the complexes into the target cells is clearly improved.

Furthermore, the transfection agent according to the present invention is characterized by simple handling thanks to the polymer/nucleic acid complexes being carried by the nanofibers. In this sense, it is favorable if the non-viral transfection agent is produced through electrospinning. The polymer/nucleic acid complexes are hereby suitable to be spun together with a carrier polymer. The resulting nanofibers are collected, by way of example, on a small glass plate and are suitable to be picked up with a pair of tweezers as a network and further processed. In this way, the network can be inserted into a cell culture vessel or directly into or onto tissue—for example in the area of a diseased organ. The subsequent processing into orally, rectally, subcutaneously, intramuscularly, intraperitoneally, or inhalatively administrable formulations is, by way of example, also conceivable. It is then particularly advantageous, inter alia, that the polymer/nucleic acid complexes are suitable to be directly released from the nanofibers onto the required spot. This significantly reduces the risk of undesired (and harmful) co-treatment of unaffected organs and body parts in comparison to an application with the aid of a simple solution.

A further advantage is that the concentration of the polymer/nucleic acid complexes in the nanofibers is very suitable to be adjusted by mixing the spinning solution together accordingly. In this way, a very exact dosage of the polymer/nucleic acid complexes is also possible. It is hereby particularly favorable that the nanofibers which carry the complexes are available in solid form and therefore, by way of example, easily weighable. It is also advantageous that the integrity of the complexes and the release of the nucleic acid from the complexes are surprisingly not affected by previous electrospinning.

It is convenient if the cationic polymer is a polyimine, preferably polyethyleneimine (PEI) or polypropyleneimine (PPI), particularly preferably polyethyleneimine (PEI). The polyethyleneimine (PEI) is hereby suitable to be a linear or branched polyethyleneimine (PEI) and/or a modified polyethyleneimine (PEI).

The cationic polymer is particularly suitable to be modified with one or several hydrophilic polymers coupled thereto. These can be selected (but not exhaustively) from the group polyethylene glycols (PEG), polyvinylpyrrolidones, polyacrylamides, polyvinyl alcohols or copolymers thereof. The hydrophilic polymer PEG is preferred.

It is also conceivable that the cationic polymer is coupled with one or several carbohydrates.

In order to achieve a cell type-specific delivery and an improved uptake of the polymer/nucleic acid complexes into the cells, it is advantageous if the cationic polymer is coupled with a receptor-specific ligand, preferably with transferrin or EGF. These ligands bind to receptors which are expressed specifically by the cells to be addressed, for example the EGF receptor. Other ligands which bind to other receptors are also conceivable, as are antibodies which recognize the surface structures of the cells and bind to them. Altogether, the ligands coupled via the cationic polymer to the polymer/nucleic acid complexes—depending on the type of ligand—are suitable to fulfill different functions, for example binding to target-cell structures on the outside of the cellular membrane, the increase in uptake efficiency for nanoscaled particles in the cells and/or the increased release of polymer/nucleic acid complexes from intracellular organelles.

The respective optimum size and type of the polymer hereby depends on the nucleic acid to be transported. Altogether, it is advantageous if the molecular weight of the cationic polymer is between 0.6 kD and 2 000 kD, preferably between 0.6 kD and 800 kD, and particularly preferably between 4 kD and 25 kD. In this range complexes from nucleic acid and polymer are obtained which are optimum with regard to complexation of the nucleic acid, biological activity/transfection efficiency and toxicity. The optimum complexation of the nucleic acid is particularly important if the polymer/nucleic acid complexes have to be processed by means of electrospinning. Since the complexation is based on electrostatic interactions, it has to comprise a certain stability to prevent itself from being destroyed in the electrical field of the spinning device or from prematurely decomposing in the aqueous medium outside the target cells. At the same time, it should not be too stable, as it would be difficult to dissolve it again within the target cells. The nucleic acids could not then become effective. Furthermore, the strength of the complex binding affects the size of the polymer/nucleic acid complex. The larger the polymer, the smaller the complex, since the DNA takes on more compact structures. However, the size of the complex is, inter alia, important for the transfection efficiency. The polymer/nucleic acid complexes preferably have a size between 20 nm and approx. 800 nm, particularly preferred between 50 nm and 500 nm.

According to the present invention, the nucleic acid in the non-viral transfection agent is suitable to be a DNA or an RNA or a DNA derivative or an RNA derivative. It is favorable if the nucleic acid is a therapeutically effective nucleic acid.

In this sense, it is conceivable that on the one hand, the nucleic acid is a DNA plasmid. This may comprise 4,000 to 10,000 bp and a gene sequence to be expressed; this is to be inserted into cell cultures with the help of the transfection system. DNAs comprising 500 up to 25,000 bp, for example smaller helper plasmids, marker-expressing plasmids or particularly large plasmids for the stable insertion of a specific gene, including control or exon/intron structures, into the target cells, are conceivable, for instance during the generation of transgenic organisms or within the context of gene therapies.

Furthermore, it is conceivable that antisense DNA or decoy DNA is also used as nucleic acids in the polymer/nucleic acid complex. These DNAs normally have less than 500 bp, and preferably less than 100 bp.

Furthermore, siRNAs, longer double-stranded RNA molecules, preferably 27-29 mers, siLNAs, sisiLNAs and siRNA molecules with other chemical modifications in order to increase stability, selectivity and/or efficiency come into particular consideration as therapeutically effective nucleic acids (though this is far from being exhaustive). Ribozymes are also conceivable, preferably Hammerhead ribozymes, though also ribozymes with Hairpin motifs or HDV ribozymes. Furthermore, messenger RNAs (mRNA) or transfer RNAs (tRNA) may be comprised as nucleic acids in the transfection agent and therefore brought into the desired cells.

The nanofibers comprise biodegradable, biocompatible polymers, preferably selected from the group polylactides, polyvinyl alcohols, polyethylene glycols, polycaprolactone, polyhydroxybutyrate, polyester urethanes, cellulose, cellulose acetate, chitosan, alginates, collagen or co-polymers and blends thereof. The composition of the polymers hereby determines the solubility of the nanofibers. It is favorable if the fibers are water-soluble.

The solubility of the nanofibers is particularly important with regard to a protracted release of the polymer/nucleic acid complexes. Depending on the nanofibers' dissolution speed, the complexes are also released quickly or slowly and therefore metered. Thus, the release kinetics of the polymer/nucleic acid complexes is suitable to be controlled through the selection of the nanofiber polymers.

This release kinetics is suitable to be additionally influenced if the entire transfection agent, namely the nanofibers including the polymer/nucleic acid complexes, is provided with an additional coating made of a biodegradable polymer, preferably with a polymer selected from the group polyethylene glycols (PEG), polyethyleneimine (PEI), poly diallyldimethylammonium chloride (PDDA), polylactide (PLA), poly-p-xylylene (PPX), copolymers and blends thereof, or lipids. This coating may, by way of example, have a thickness of 10 nm up to several micro meters.

It is a matter of course that the size of the nanobfibers plays a role for the amount of polymer/nucleic acid complexes released in total. This is, by way of example, suitable to be an average ranging from 1nm to 100 μm, preferably between 10 nm and 10 μm, and particularly preferably between 50 nm and 3 μm. The length of the fibers is suitable, depending on the application, to be between approx. 2 μm and 100 μm; other dimensions such as those in the centimeter range and also depending on the application are conceivable. A particular advantage of the production method for the non-viral transfection agent according to the present invention is that the polymer/nucleic acid complexes are electrospun together with the carrier polymers to the nanofibers. As such, only the provision of polymer/nucleic acid complexes and the provision of a spinning solution for preparation are necessary.

The provision of the polymer/nucleic acid complex comprises the following steps
production of a nucleic acid solution
incubation of the nucleic acid solution
production of a polymer solution
incubation of the polymer solution
addition of the polymer solution to the nucleic acid solution
mixing
incubation of the mixture

As a rule, this procedure does not take longer than two hours and is therefore significantly less time-consuming and complicated than the preparation of a virus for transfection. Practical embodiment 1 as described below shows an example protocol for the production of PEI-F25-LMW polymer/nucleic acid complexes.

The provision of the spinning solution is not particularly time-consuming either. It comprises the following steps according to the present invention:
provision of a solution comprising polymer/nucleic acid complexes in a suitable medium;
provision of a solution of the carrier polymer, preferably an aqueous solution;
addition of the polymer/nucleic acid complex solution to the carrier polymer solution;
mixing.

It should be noted that the first step, namely the provision of the solution comprising polymer/nucleic acid complexes, will, in practice, usually already be the result of the provision of the polymer/nucleic acid complex, as the provision of a polymer/nucleic acid complex takes place appropriately in a suitable medium, e.g. in ‘HEPES buffer’ (10 mM to 1 M HEPES, 150 mM NaCl, pH 7.4).

Electrospinning takes places as a further step.

Altogether, it can be recognized that the non-viral transfection agent is not only able to be stored effectively, but is also suitable to be produced with simple means and in a proportionately cost-effective manner. Furthermore, the nucleic acids are protected against degradation through the complexation of the nucleic acids with cationic polymers. The loco-regionally controlled and temporally controlled release of the nucleic acids is particularly facilitated through being carried by the nanofibers. In addition, the simple handling of the solid-like transfection agent is advantageous.

All of these factors are particularly favorable with regard to the use of the transfection agent. As such, the non-viral transfection agent according to the present invention is suitable for use for the transfection of eucaryotic cells. By way of example, applications both in the cell culture laboratory and directly in the organism are thereby conceivable. In looking at the latter option, the non-viral transfection agent is suitable to be used to produce a pharmaceutical.

A pharmaceutical comprising a non-viral transfection agent according to the present invention is, in turn, advantageously characterized by simple handling and loco-regional delivery of therapeutic nucleic acids which can be controlled effectively. In this sense, a protracted release is also good and easily controllable.

The use of such a pharmaceutical is therefore appropriate for a gene therapy treatment. As the non-viral transfection agent according to the present invention facilitates a multitude of formulations and forms of administration without problems, the pharmaceutical is suitable to be adjusted to the corresponding individual treatment in an entirely targeted manner.

Further characteristics, details and advantages of the invention derive from the wording of the claims, as well as from the following description of practical embodiments on the basis of the figures. These show:

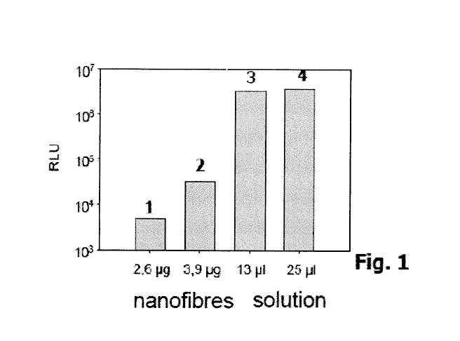

FIG. 1 transfection efficiency of spun nanoplexes according to practical embodiment 4,

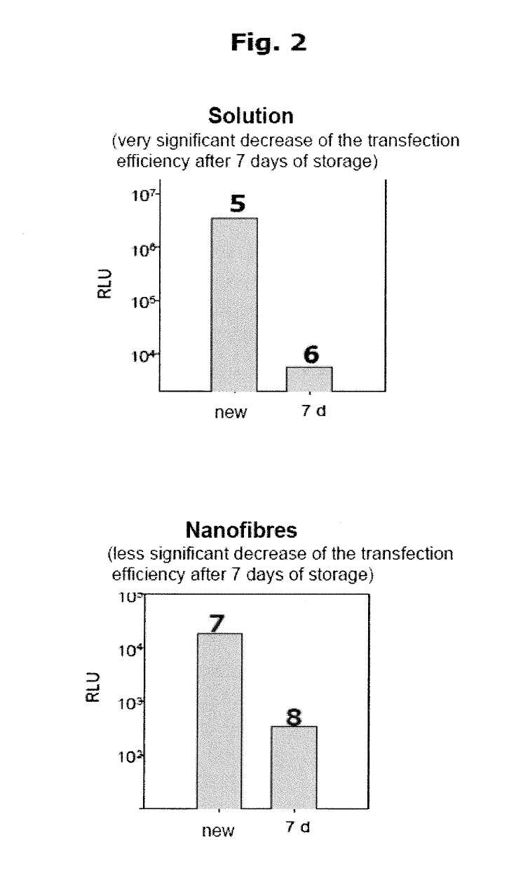

FIG. 2 storage of spun nanoplexes according to practical embodiment 5,

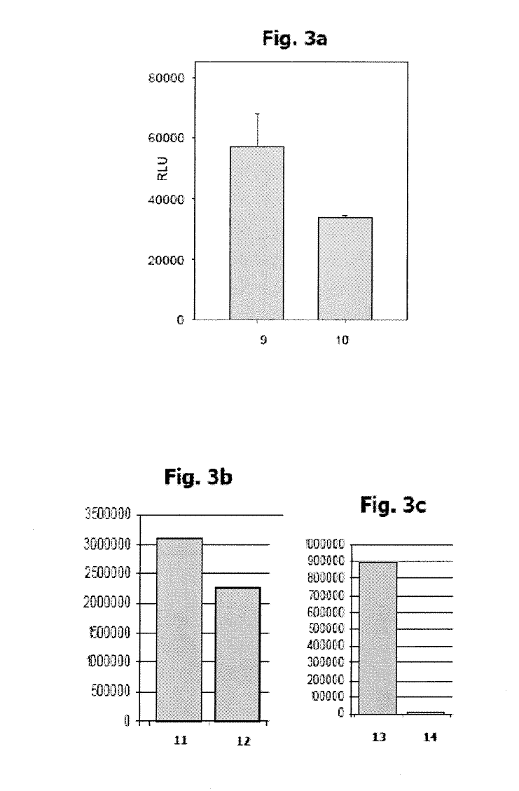

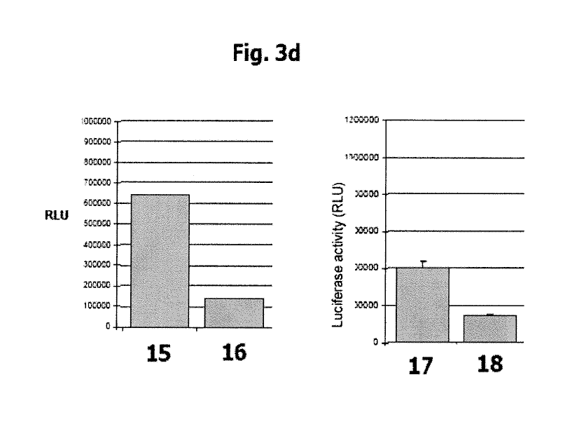

FIG. 3a knockdown efficiency (PEO solution) according to practical embodiment 6a,

FIG. 3b knockdown efficiency (PEO/dichloromethane solution) according to practical embodiment 6b,

FIG. 3c knockdown efficiency (PVA solution) according to practical embodiment 6c, and

FIG. 3d knockdown efficiency (PEO/water solution) according to practical embodiment 6d.

### Practical Embodiments

### Practical Embodiment 1—Production of Polymer/Nucleic Acid Complexes (PEI-F25-LMW)

a) For the production of a polymer/nucleic acid complex for the direct and new application or freezing, the known production of PEI F25-LMW/DNA complexes has to be mentioned here by way of example.

For that purpose, 1.3 μg DNA is dissolved in 80 μl HEPES buffer (10 mM to 1M HEPES, 150 mM NaCL, pH 7.4) and incubated for 10 minutes.

22 μl PEI F25-LMW (0.6 μg/μl) is dissolved in 80 μl HEPES buffer (10 mM to 1M HEPES, 150 mM NaCL, pH 7.4) and after these 10 minutes pipetted to the DNA solution.

The complexation takes place through incubation for up to 1 hour at room temperature. The complexes are then suitable to be aliquoted and frozen. The complexes only have to be thawed for further processing. When the complexes have been thawed, they are mixed via brief vortexing and incubated again for 30-60 min.

Depending on the type and size of the nucleic acid to be complexed, all indications of amount and volume, as well as the mixing ratio of the polymer and nucleic acid solution, are suitable to be adjusted to the respective requirements. The produced total volume of the complex solution also has to be understood as exemplary and is also suitable, as a matter of course, to be produced on a midi or maxi scale or industrial scale, respectively.

b) For the production of a polymer/nucleic acid complex for lyophilization, the known production of PEI F25-LMW/DNA complexes has to be mentioned by way of example.

For this purpose, 260 μg DNA is dissolved in 1,040 μl of 5% glucose in water and incubated for 10 minutes. 213 μl PEI F25-LMW (6.1 μg/μl) is dissolved in 1,040 μl of 5% glucose and after these 10 minutes pipetted to the DNA solution. After brief vortexing, it is incubated for 1 h and then briefly vortexed again.

26 aliquots of 92 μl each are produced and lyophilized under standard conditions (reduced temperature, vacuum). The remainders are suitable to be taken up again in different aqueous or organic solvents.

The following also applies: Depending on type and size of the nucleic acid to be complexed, all indications of amount and volumes, as well as the mixing ratio of the polymer and the nucleic acid solution, are suitable to be adjusted to the respective requirements. The total volume of the complex solution produced also has to be understood by way of example and is suitable, as a matter of course, to be produced also on a midi or maxi scale or industrial scale, respectively.

### Practical Embodiment 2—Production of the Spinning Solution

For the production of the spinning solution, between 0.2 and 20 wt.-% of the polymer to be spun is dissolved in a solvent. Water preferably comes into consideration as a solvent, though organic solvents, e.g. dichloromethane or hexafluoroisopropanole, are also considered. The selection depends on the selected carrier polymer.

### Practical Embodiment 3—Electrospinning, Production of the Transfection Agent for siRNA

For immobilization, 165 μg of the PEI/siRNA complexes in each case (with a luciferase-specific or non-specific siRNA) was dissolved in 100 μL HEPES buffer (10 mM HEPES, 150 mM NaCl), thawed and added to 200 μL of a 6 wt.-% PEO/water solution. After mixing in an orbital shaker (2,500 rpm), the spinning solution was spun at a voltage of approx. 17 kV and at a distance of 14 cm on small glass plates as carrier matrix. The flow rate was 0.2 ml/h.

### Practical Embodiment 4—Transfection Efficiency of Spun Nanoplexes in Comparison to Solutions

In FIG. 1 it can be recognized that with the help of the transfection agent according to the present invention, a significantly more precise control of the transfection efficiency can be achieved and therefore of the dosage of the active agent to be introduced into the cells than with conventional transfection solutions.

In the experiment represented, 100,000 ovarian carcinoma SKOV-3 cells/well are seeded in a six-well plate and cultured for 24 h in IMDM/10% FCS medium. By way of example, between 1 μg and 10 μg nanofibers with PEI/luciferase DNA complexes carried thereon is then removed with tweezers from the small glass carrier plate or rinsed off with liquid and added to the medium. Alternatively, the small glass plates with the applied nanofibers are also suitable to be placed in an inverted manner into the well on the medium. The measurement of the the luciferase activity typically takes place between 48 h and 96 h in the luminometer after insertion of the nanofibers by means of chemiluminescence.

Columns 1 and 2 show that a significant difference occurs with regard to the transfection efficiency if the inserted amount of DNA is varied when dealing with a transfection with the help of the transfection agent according to the present invention. As such, 2.6 μg DNA was used in column 1 and 3.9 μg DNA in column 2. The resulting luminescence—and thus the effect of the luciferase plasmids that arrived in the cells—differs by an order of magnitude between both transfections. If, in contrast, the amount of DNA is varied in a traditional way, e.g. by multiplying the amount of added solution, then columns 3 and 4 show that almost no difference in the transfection efficiency occurs. It can clearly be recognized that the transfection agent according to the present invention thus allows for a significantly improved control and dosage of the delivery of the nucleic acid active agent.

### Practical Embodiment 5—Storage Capabilities of Spun Nanoplexes

It can be recognized in FIG. 2 that a further advantage of the transfection agent according to the present invention is the improved storage capabilities. As a result of this, it is conceivable, by way of example, for the nanofibers comprising polymer/nucleic acid complexes to be processed into a form of administration such as tablets, thereby ensuring that the transfection agent as a therapeutic agent is as easy to administer as possible to a patient.

The nanofibers with spun nanoplexes are stored in a dry form at 4° C. or RT over a prolonged period of time, typically several weeks. The measurement of the transfection efficiency takes place as in practical embodiment 4.

While the transfection efficiency of the conventionally used solution decreases by more than two magnitudes during storage over a period of approximately 7 days, a reduction of only slightly more than one magnitude can be detected in the polymer/nucleic acid complexes spun in nanofibers according to the present invention.

Practical embodiments 6a to 6d, whose results are represented in FIGS. 3a to 3d, furthermore show that a targeted knockdown of genes is possible with the help of the transfection agent according to the present invention by inserting siRNA. Different spinning solutions (examples 6a to 6d, FIGS. 3a to 3d) are hereby suitable on the one hand and the nanofibers obtained are capable of being additionally provided with a polymeric coating on the other (cf. example 6d, FIG. 3d).

### Practical Embodiment 6a—Knockdown Efficiency of PEI/siRNA Complexes Which are Electrospun in PEO Solution

PEI/siRNA complexes are produced as described in practical embodiment 1. These complexes are spun according to practical embodiment 3. The transfection agent obtained in this way is used for the following experiment:

40,000 stably luciferase expressing SKOV-3/Luc ovarian carcinoma cells/well are seeded into a 24 well plate and cultured for 24 h in IMDM/10% FCS medium. By way of example, between 1 μg and 10 [g nanofibers with PEI/luciferase siRNA complexes carried thereon is then removed with tweezers from the small glass carrier plate or rinsed off with liquid and added to the medium. These can, for example, be PEO nanofibers with 0.63 μg complexed luciferase siRNA or a non-specific control siRNA. The measurement of the luciferase activity typically takes place between 48 h and 96 h in the luminometer after insertion of the nanofibers by means of chemiluminescence.

In order to control the specificity, one specific and one non-specific siRNA are complexed in parallel assays and applied to a carrier. The targeting efficiency is calculated from the percentage decrease of the luciferase activity of the cells treated with PEI/spec. siRNA complexes carried by nanofibers versus the luciferase activity of the cells treated with PEI/non-spec. siRNA complexes.

In FIG. 3a it can be recognized that the chemiluminescence of the cells treated with specific siRNA decreases significantly in comparison to the cells treated with non-specific siRNA. Column 9 hereby shows the chemiluminescence caused by the luciferase expression of the cultured cells which were treated with the control siRNA. Column 10 shows decreased chemiluminescence due to the treatment with specific luciferase siRNA. Due to the treatment with the control siRNA, non-specific effects such as cytotoxic effects are excluded as the cause for the gene knockdown.

### Practical Embodiment 6b—Knockdown Efficiency of PEI/siRNA Complexes Which are Electrospun in PEO/Dichloromethane Solution

PEI/siRNA complexes are produced as described in practical embodiment 1.

For the production of fibers spun from dichloromethane, the PEI/siRNA complexes lyophilized in glucose solution were suspended with a PEO/DCM solution with a concentration of 1 wt.-%. The solution obtained was processed at 7.5 kV and at a distance of approx. 14 cm between the electrodes, and applied on small glass plates as a carrier matrix.

The knockdown efficiency was determined according to practical embodiment 6a.

As in practical embodiment 6a and FIG. 3a, respectively, it can also be recognized here that in contrast to the non-specific control RNA, the specific luciferase siRNA causes a knockdown of the luciferase expression. Column 11 hereby shows the luminescence caused by the luciferase expression of the cultured cells which were treated with the control siRNA. Column 12 shows decreased luminescence due to the treatment with specific luciferase siRNA.

### Practical Embodiment 6c—Knockdown Efficiency of PEI/siRNA Complexes Which are Electrospun in PVA Solution

PEI/siRNA complexes are produced as described in practical embodiment 1. These complexes are further processed through electrospinning.

For immobilization purposes, 165 μg of the PEI/siRNA complexes in each case (both of the luciferase-specific and the non-specific siRNA) was dissolved in 100 μL HEPES buffer (10 mM HEPES and 150 mM NaCl), thawed and added to 200 μL of a 12 wt.-% PVA/water solution. After mixing in an orbital shaker (2,500 rpm), the spinning solution was spun at a voltage of approx. 21 kV and at a distance of 14 cm on small glass plates as a carrier matrix. The flow rate was 0.15 ml/h.

The knockdown efficiency was determined according to practical embodiment 6a.

As shown in practical embodiments 6a and 6b and FIG. 3a and FIG. 3b, respectively, it can also be recognized here that in contrast to the non-specific control RNA, the specific luciferase siRNA causes a knockdown of the luciferase expression. Column 13 hereby shows the luminescence caused by the luciferase expression of the cultured cells which were treated with the control siRNA. Column 14 shows decreased luminescence due to the treatment with specific luciferase siRNA.

### Practical Embodiment 6d—Knockdown Efficiency After Four and After Seven Days of PEI/siRNA Complexes Which are Electrospun in PEO/Water Solution, Wherein the Fibers are Coated with PPX

PEI/siRNA complexes are produced as described in practical embodiment 1. These are electrospun as described in practical embodiment 3.

For the PPX coating of the PEO fibers charged with PEI/siRNA complex (refer to practical embodiment 3), the coater Labcoater 1 PDS 2010 from the company SPECIALTY COATING SYSTEMS was used for chemical vapor deposition (CVD). The fiber mats were fixed to a bent metal bar, which was attached to the floor of the coating device. With a weighing amount of [2.2]paracyclophane of 100 mg, PPX coatings of a thickness of approximately 50 nm and of 1 g of approx. 500 nm were produced.

The knockdown efficiency was determined according to practical embodiment 6a.

As in practical embodiments 6a and 6c and FIG. 3a and FIG. 3c, respectively, it can also be recognized here that in contrast to the non-specific control RNA, the specific luciferase siRNA causes a knockdown of the luciferase expression.

Column 15 hereby shows the luminescence caused four days after the transfection by the luciferase expression of the cultured cells which were treated with the control siRNA. Column 16 shows decreased luminescence in the same period of time due to the treatment with specific luciferase siRNA. In contrast, column 17 and column 18 show the corresponding luminescence seven days after the transfection, wherein column 17 in turn shows the luminescence caused by the luciferase expression of the cultured cells which had been treated with the control siRNA, and column 18 shows decreased luminescence through the treatment with specific luciferase siRNA.

It can hereby be recognized in FIG. 3d that the reduction of the luciferase expression, which is associated with the knockdown, is suitable to be clearly detected after just 4 days. Afterwards, it remains on a constantly low level (cf. column 16 and column 18, effect after 4 and after 7 days). This shows that the transfection agent according to the present invention allows for a continuous effect over a longer period of time.

The invention is not limited to one of the previously described embodiments; it is suitable for being modified in all kinds of ways. Thus, it is conceivable, by way of example, that ligands are coupled to the nanofibers after electrospinning.

The solubility of the polymer/nucleic acid complexes is suitable to be influenced by the additional deposition of an outer layer, e.g. a layer made of lipids.

It is also conceivable that several different nucleic acids are inserted into one complex, e.g. expression and helper plasmids. Alternatively, it is possible to spin several different complexes simultaneously.

It can be recognized that a non-viral transfection agent advantageously comprises polymer/nucleic acid complexes and nanofibers, wherein the polymer/nucleic acid complexes comprise at least one nucleic acid and at least one cationic polymer. The nanofibers carry the polymer/nucleic acid complexes, wherein the non-viral transfection agent is appropriately produced through electrospinning.

Furthermore, it can be recognized that the cationic polymer is favorably a polyimine, preferably polyethyleneimine, and is suitable to be modified with one or several hydrophilic polymers coupled thereto. It can also be appropriate to couple the cationic polymer with one or several carbohydrates and/or with one receptor-specific ligand. The nucleic acid is a DNA or RNA, or a DNA derivative or an RNA derivative, advantageously a therapeutically effective nucleic acid. The nanofibers comprise biodegradable, biocompatible polymers. The fibers, or rather the entire transfection agent, are suitable to be provided with a polymeric coating.

Furthermore, it can be recognized that a method for the production of a non-viral transfection agent comprises the following steps: provision of the polymer/nucleic acid complex, production of a spinning solution comprising the polymer/nucleic acid complexes, and electrospinning.

Furthermore, it can be recognized that the non-viral transfection agent is suitable to be used for the transfection of eukaryotic cells and for the production of a pharmaceutical. The pharmaceutical is suitable to be used advantageously for gene therapy treatments.

The present invention therefore refers to a non-viral transfection agent, comprising polymer/nucleic acid complexes and nanofibers, wherein the polymer/nucleic acid complexes comprise at least one nucleic acid and at least one cationic polymer. The nanofibers carry the polymer/nucleic acid complexes, wherein the non-viral transfection agent is appropriately produced through electrospinning. The cationic polymer is favorably a polyimine or polyethyleneimine, respectively, and is suitable to be modified with one or several hydrophilic polymers coupled thereto. It can also be appropriate to couple the cationic polymer with one or several carbohydrates and/or with one receptor-specific ligand. The nucleic acid is a DNA or RNA, or a DNA derivative or an RNA derivative, advantageously a therapeutically effective nucleic acid. The nanofibers comprise biodegradable, biocompatible polymers. The fibers, or rather the entire transfection agent, are suitable to be provided with a polymeric coating. A method for the production of a non-viral transfection agent comprises the following steps: provision of the polymer/nucleic acid complex, production of a spinning solution comprising the polymer/nucleic acid complexes, and electrospinning.

It can be recognized that the non-viral transfection agent is suitable to be used for the transfection of eukaryotic cells and for the production of a pharmaceutical. The pharmaceutical is suitable to be used advantageously for gene therapy treatments.

All of the characteristics and advantages originating from the claims, description and figures, including constructive details, spatial arrangements and processing steps, are suitable for being essential to the invention, both in themselves and in the most diverse combinations.

1. A non-viral transfection agent comprising polymer/nucleic acid complexes and nanofibers, wherein the polymer/nucleic acid complexes comprise at least one nucleic acid and at least one cationic polymer.

2. The non-viral transfection agent according to claim 1, wherein the nanofibers carry the polymer/nucleic acid complexes.

3. The non-viral transfection agent according to claim 1, wherein the non-viral transfection agent is produced through electrospinning.

4. The non-viral transfection agent according to claim 1, wherein the cationic polymer is a polyimine.

5. The non-viral transfection agent according to claim 1, wherein the cationic polymer is modified with at least one hydrophilic polymer coupled thereto.

6. The non-viral transfection agent according to claim 1, wherein the cationic polymer has a molecular weight between 0.6 kD and 2 000 kD.

7. The non-viral transfection agent according to claim 1, wherein the nucleic acid is selected from the group consisting of a DNA, an RNA, a DNA derivative, and an RNA derivative.

8. The non-viral transfection agent according to claim 1, wherein the nucleic acid is a therapeutically effective nucleic acid.

9. Non-viral transfection agent according to claim 1, wherein the nanofibers comprise biodegradable, biocompatible polymers.

10. The non-viral transfection agent according to claim 1, wherein the transfection agent further comprises a polymeric coating.

11. The non-viral transfection agent according to claim 1, wherein the nanofibers have a diameter between 1 nm and 100 μm.

12. A method for the production of a non-viral transfection agent comprising polymer/nucleic acid complexes and nanofibers, wherein the polymer/nucleic acid complexes comprise at least one nucleic acid and at least one cationic polymer, the method comprising:
providing the polymer/nucleic acid complexes;
producing a spinning solution comprising the polymer/nucleic acid complexes and at least one carrier polymer; and
electrospinning the spinning solution.

13. A method of transfecting a eukarvotic cell, the method comprising exposing the eukarvotic cell to a non-viral transfection agent comprising nanofibers and polymer/nucleic acid complexes comprising at least one nucleic acid and at least one cationic polymer.

14. (canceled)

15. (canceled)

16. (canceled)

17. The non-viral transfection agent according to claim 4, wherein the polyimine is polyethyleneimine (PEI) or polypropyleneimine (PPI).

18. The non-viral transfection agent according to claim 17, wherein the polyimine is polyethyleneimine (PEI).

19. The non-viral transfection agent according to claim 6, wherein the molecular weight of the cationic polymer is between 0.6 kD and 800 kD.

20. The non-viral transfection agent according to claim 19, wherein the molecular weight of the cationic polymer is between 4 kD and 25 kD.

21. The non-viral transfection agent according to claim 9, wherein the biodegradable, biocompatible polymers are selected from the group consisting of polylactides, polyvinyl alcohols, polyethylene glycols, polycaprolactone, polyhydroxybutyrate, polyester urethanes, cellulose, cellulose acetate, chitosan, alginates, collagen, and co-polymers and blends thereof.

22. The non-viral transfection agent according to claim 10, wherein the polymeric coating comprises a polymer selected from the group consisting of polyethylene glycols (PEG), polyethyleneimine (PEI), poly diallyldimethylammonium chloride (PDDA), polylactides (PLA), polycaprolactone (PCL), polyester urethanes (PEU), cellulose, cellulose acetate, chitosan, alginate, collagen, poly-p-xylylene (PPX), and lipids, copolymers, and blends thereof.

23. The non-viral transfection agent according to claim 22, wherein the polymer is biodegradable.

24. The non-viral transfection agent according to claim 11, wherein the diameter of the nanofibers is between 10 nm and 10 μm.

25. The non-viral transfection agent according to claim 24, wherein the diameter of the nanofibers is between 50 nm and 3 μm.
